(19) United States
(12) Patent Application Publication
(10) Pub. No.: US 2008/0243025 A1
(43) Pub. Date: Oct. 2, 2008

(54) Medical Device

(76) Inventors: Nils Holmstrom, Jarfalla (SE); Malin Ohlander, Stockholm (SE); Sven Kalling, Taby (SE)

Correspondence Address: SCHIFF HARDIN, LLP;PATENT DEPARTMENT, 6600 SEARS TOWER, CHICAGO, IL, 60606-6473, US

(21) Appl. No.: 11/793,849

(22) PCT Filed: Dec. 23, 2004
(86) PCT No.: PCT/SE2004/002022

§ 371 Date: Dec. 3, 2007

(51) Int. Cl. A61B 5/053

(52) U.S. Cl. 600/547

(57) ABSTRACT

In a medical device and a method for operating the medical device, it is first determined whether a patient, at whom a medical measurement is to be made, satisfied specified criteria that will ensure comparability of the measurement results obtained from the patient. Only when the specified criteria had been satisfied is an electrical bio-impedance signal obtained from the patient. The cardiac component of the electrical bio-impedance signal is extracted, and is analyzed to identify a change in a medical condition of the patient.

### BACKGROUND OF THE INVENTION

1. Field of the Invention

The present invention generally relates to implantable medical devices, such as cardiac pacemakers and implantable cardioverter/defibrillators, and in particular to method and medical device for obtaining electrical bio-impedance signals in order to monitor or detect changes in a condition of the heart of a patient.

Today, in the modern society, heart diseases and/or conditions leading to an impaired heart function are a major problem entailing constantly increasing costs for medical services. For example, heart failure is a condition which affects thousands of people throughout the world. Congestive heart failure (CHF) is an inability of the heart to pump blood at an adequate rate in response to the filling pressure. Patients suffering from CHF are often afflicted by cardogenic pulmonary edema, which is caused by the accumulation of fluid in the lung interstitium and alveoli due to the fact the left ventricular venous return exceeds left ventricular cardiac output. That is, more fluids are transported to the lung region than from the lung region causing the accumulation of fluids in the lung region. CHF may even, in its more severe stages, result in death.

The progression of fluid accumulation in pulmonary edema, whether it is initiated by damage to the various components of the alveolar-capillary membranes or is cardiogenic in nature, can be identified by three distinct physiological stages. Stage I: Fluid and colloid shift into the lung interstitium from pulmonary capillaries, but an increase in lymphatic outflow efficiently removes the fluid. Stage II: The continuing filtration of liquid and solutes overpowers the pumping capacity of the lymphatic system. The fluid initially collects in the more compliant interstitial compartment. Stage III: As fluid filtration continues to increase and the filling of loose interstitial space occurs, fluid accumulation in the less compliant compartment takes place. In certain cases, the interstitial space may contain up to 500 ml of fluid. Eventually, if the accumulation continues, the fluid may cross the alveolar epithelium in to the alveoli, leading to alveolar flooding. Hence, incipient pulmonary edema is an effective indicator of worsening CHF.

Furthermore, many heart diseases can also be identified by detecting changes of certain variables or parameters indicative of different functions of the heart, such as the systolic and diastolic slopes, pre-ejection period and left ventricular ejection time.

Electrical bio-impedance signals has been found to be an effective measure for identifying changes of many different conditions in the body of a patient, such as incipient pulmonary edema and the progression of pulmonary edema due to CHF. For example, the accumulation of fluids in the lung-region associated with pulmonary edema affects the thoracic impedance, or more specifically the DC impedance level, since the resistivity of the lung changes in accordance with a change of the ratio of fluid to air. The DC impedance level is negatively correlated with the amount of fluids in the lung. Studies have shown that hospitalization due to the development of acute CHF with the symptom pulmonary edema was preceded two or three weeks by a drop in the DC impedance by approximately 10-15%.

In addition to the thoracic impedance, the cardiogenic impedance, which is defined as the impedance or resistance variation that origins from cardiac contractions measured by electrodes inside or on the surface of the body, can be used for identifying changes of different conditions in the heart of a patient. For example, parameters such as the systolic and diastolic slopes, pre-ejection period and left ventricular ejection time indicative of different functions of the heart can be extracted from the cardiogenic impedance. The impedance is calculated as z=u/i, where u is the measured voltage between two electrodes and i is the applied excitation current between the two electrodes. The electrodes are placed inside or on the surface of the heart, integrated on a pacemaker lead or outside of the heart such as the pacemaker encapsulation. The cardiogenic impedance variation correlates to the volume changes of the heart chambers, which can be used as an indication of the dynamic blood filling. Hence, changes of these parameters due to a change in the heart, for example, caused by a disease such as heart failure can be detected by monitoring or detecting changes of the cardiogenic impedance. Several different impedance measurement configurations are known. In the most basic configuration the measurement current is injected between two electrodes and the voltage is measured between the same electrodes. The impedance is calculated as u/i. Since the impedance value is significantly affected by the tissue resistivity near the current injecting electrodes other impedance measurement configurations have been developed. The tripolar configuration uses one current injecting electrode and one voltage measurement electrode and one common electrode used for both current injection and voltage measurement. One example of such an arrangement is a configuration where the measurement current is injected between a pacemaker encapsulation and a pacing electrode tip while the voltage is measured between the pacing electrode ring or indifferent electrode and the pacemaker encapsulation. This configuration has the advantage that it improves the measurement sensitivity for tissue resistivity variations for tissue located at some distance from the electrodes used for impedance measurement. This configuration, referred to as tripolar configuration, improves the sensitivity for pulmonary edema monitoring. In a further improvement two separate electrodes are used for current injection and two separate electrodes are used for voltage measurement. This last configuration is commonly referred to as quadropolar configuration.

Accordingly, an effective method for measuring or detecting changes in electrical bio-impedances, such as the intra thoracic impedance or the cardiogenic impedance, i.e. the cardiac component of an impedance signal measured over the heart, would be of a great value. However, a problem associated with such measuring methods is the accurateness and reliability of the obtained signals since they are greatly affected by factors like the body position of the patient, patient activity levels, heart rate frequency, etc. For example, it has been found that the body position of the patient is of major importance with regard to the thoracic impedance as well as the cardiogenic impedance. Moreover, it has also been found that the heart rate frequency has a major impact on the cardiogenic impedance. A number of attempts to eliminate or filter out these error sources have therefore been proposed. For example, U.S. Pat. No. 6,104,949 discloses a method and device for treatment of CHF, in which changes in the posture of the patient is correlated with changes of the trans-thoracic impedance. A posture sensing means indicates whether the patient lies down or is standing and the measurement of the trans-thoracic impedance is then correlated with periods when the patient is lying down or standing up.

However, is has recently been found that the position dependence also is of a significant magnitude regarding different positions even when the patient is lying down, for example, whether the patient is lying on a side or is lying on the back. A major reason is that an impedance measurement depends on the measurement vector, i.e. the vector between the nodes that the current is applied between and the vector the voltage is measured between. When the body shifts position, these vectors will change since the gravity will influence, for example, tissue between the nodes and how it moves. Tests performed on animals have shown that the trans thoracic impedance may vary up to 20% depending on which position the animal was lying in.

Accordingly, there is a need of an improved method and medical device that are able to obtain electrical bio-impedance signals in order to monitor or detect changes of a condition of a patient in a more reliable and accurate manner.

### SUMMARY OF THE INVENTION

An object of the present invention is to provide an improved method and medical device that are able to obtain electrical bio-impedance signals in order to monitor or detect changes of a condition of a patient in a more reliable and accurate manner

This and other objects are achieved according to the present invention by a method, medical devices, and a computer readable medium wherein an electrical bio-impedance is measured at a patient, the electrical bio-impedance being associated with a medical condition of the patient, and the cardiac component of the electrical bio-impedance is measured. Before measuring the electrical bio-impedance at the patient, it is determined that the patient satisfies specified criteria ensuring comparable measurement results and, when said criteria have been satisfied, the measurement of the electrical bio-impedance is initiated, in order to obtain substantially repeatable impedance signals. The impedance signals are analyzed to identify a change in the medical condition from the impedance signals.

The measured impedance has a DC component and an AC component, the DC component being the baseline around which the AC component fluctuates. The DC component reflects the amount of tissue and fluids that are located between the measuring points that the impedance is measured in-between and the AC component reflects how respiration and cardiac activity influence the impedance signal.

As used herein, the term “intra thoracic impedance” refers to an impedance measurement over the thorax by using an implantable medical device, i.e. an impedance measurement where the impedance measurement vector spans over the thorax.

Moreover, the term “cardiac component of the electrical bio-impedance” as used herein is defined as the impedance or resistance variation that origins from cardiac contractions or, in other words, the cardiac component of the impedance measured between electrodes within the heart.

According to an embodiment of the present invention, a method for detecting a change of a condition of a patient includes detecting a position of the patient; and measuring the impedance arranged to sense an electrical bio-impedance associated with the condition. A specific body position of the patient Is detected and an impedance measuring session is initiated in order to obtain substantially repeatable impedance signals, wherein a change of said condition can be derived from said impedance signals.

According to a second embodiment of the present invention, a medical device for detecting a change of a condition of a patient has a position detector that detects a position of the patient; and an impedance measuring arrangement that measures an electrical bio-impedance associated with the condition. The device has a position detector that detects a predetermined, specific body position of the patient and, when detecting that the patient is in the specific body position, the position detects or supplies a triggering signal to the arrangement, which impedance measuring upon receiving the triggering signal, initiates an impedance measuring session in order to obtain substantially repeatable impedance signals, wherein a change of the condition can be derived from the impedance signals.

According to a third embodiment of the present invention, a computer readable medium is encoded with a data structure that represents instructions for causing a computer to perform a method according to the first aspect.

Thus, the invention is based on measuring the electrical bio-impedance only when the patient is in a predetermined specific body position. By performing the impedance measurement only in this specific position, impedance signals that are substantially repeatable can be obtained. In this manner, changes of a condition of the patient or trends in the development of a condition of a patient can be monitored or detected in an effective way.

This solution provides several advantages over the existing solutions. One advantage is that the obtained signals are very accurate and reliable since the measurements are performed only when the patient is in a predetermined specific body position. This entails that variations in the signals due to measurements in different body positions can be substantially eliminated, which is an evident risk with the method disclosed in U.S. Pat. No. 6,104,949 where the impedance measurements is correlated with moments when the patient is lying down and, therefore, the measurements are, in practical, performed in a number of different positions, i.e. when the patient is lying on either side or when the patient is lying on the back, etc.

Another advantage is that the measurements are initiated only when the patient is in the specific predetermined position whereby a more efficient method with respect to current consumption is achieved in comparison with the method according to U.S. Pat. No. 6,104,949 where the impedance measurements are performed on a constant basis and when it is detected that the patient is lying down the measurement values for the assessing of the degree of heart failure are obtained and stored.

In accordance with one embodiment of the present invention, the specific body position when the patient is lying on the back.

In one embodiment the intra thoracic impedance is sensed. This allows the progression of pulmonary edema can be monitored since the accumulation of fluids in the lung-region associated with pulmonary edema affects the thoracic impedance, or more specifically the DC impedance level, since the resistivity of the lung changes in accordance with a change of the ratio of fluid to air. The DC impedance level is negatively correlated with the amount of fluids in the lung. Thus beginning pulmonary edema can be detected through DC impedance measurements. For example, studies have shown that hospitalization due to the development of acute CHF with the symptom pulmonary edema was preceded two or three weeks by a drop in the DC impedance by approximately 10-15%.

According to another embodiment the sensed intra thoracic impedance is used to detect incipient pulmonary edema.

According to a further embodiment the sensed intra thoracic impedance is used to detect the development of the pulmonary edema after the patient has been hospitalized and the patient is improving the pulmonary edema situation.

Alternatively, the cardiac component of the electrical bio-impedance is sensed, which can be used for identifying changes different conditions in the heart of a patient.

The cardiac component of the electrical bio-impedance can be used to extract surrogates of the heart function from the group of: systolic and diastolic slopes, the pre-ejection period, or the left ventricular ejection time.

### BRIEF DESCRIPTION OF THE DRAWINGS

FIG. 1 is schematic diagram showing a medical device implanted in a patient with which the present invention can be implemented.

FIG. 2 is block diagram of the basic functional components of a first embodiment of the present invention.

FIGS. 3a, 3b, and 3c are schematic diagrams of a first embodiment of the position detecting sensor of FIG. 1.

FIG. 4 is a flow chart illustrating the steps in accordance with one embodiment of the present invention to measure the electrical bio-impedance indicative of changes of a condition of the patient or trends in the development of a condition of a patient.

### DESCRIPTION OF THE PREFERRED EMBODIMENTS

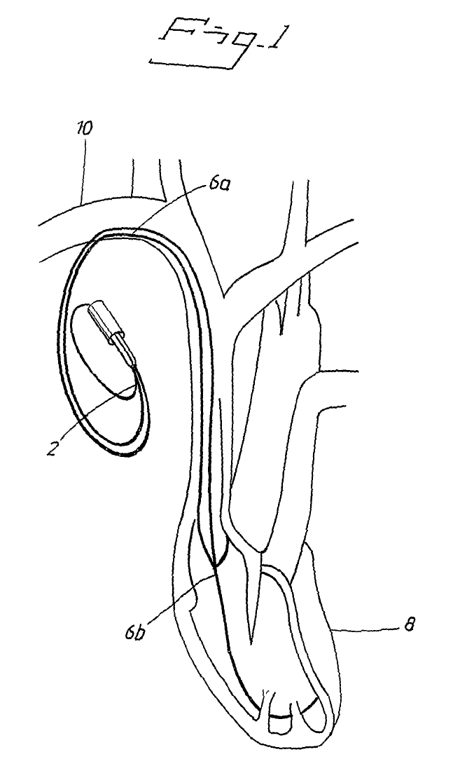

FIG. 1 shows a schematic diagram of a medical device implanted in a patient in which device the present invention can be implemented. As can be seen, this embodiment of the present invention is shown in the context of a pacemaker 2 implanted in a patient (not shown). The pacemaker 2 comprises a housing being hermetically sealed and biological inert. Normally, the housing is conductive and may, thus, serve as an electrode. One or more pacemaker leads, where only two are shown in FIG. 1 namely a ventricular lead 6a and an atrial lead 6b, are electrically coupled to the pacemaker 2 in a conventional manner. The leads 6a, 6b extend into the heart 8 via a vein 10 of the patient. One or more conductive electrodes for receiving electrical cardiac signals and/or for delivering electrical pacing to the heart 8 are arranged near the distal ends of the leads 6a, 6b. As the skilled man in the art realizes, the leads 6a, 6b may be implanted with its distal end located in either the atrium or ventricle of the heart 8.

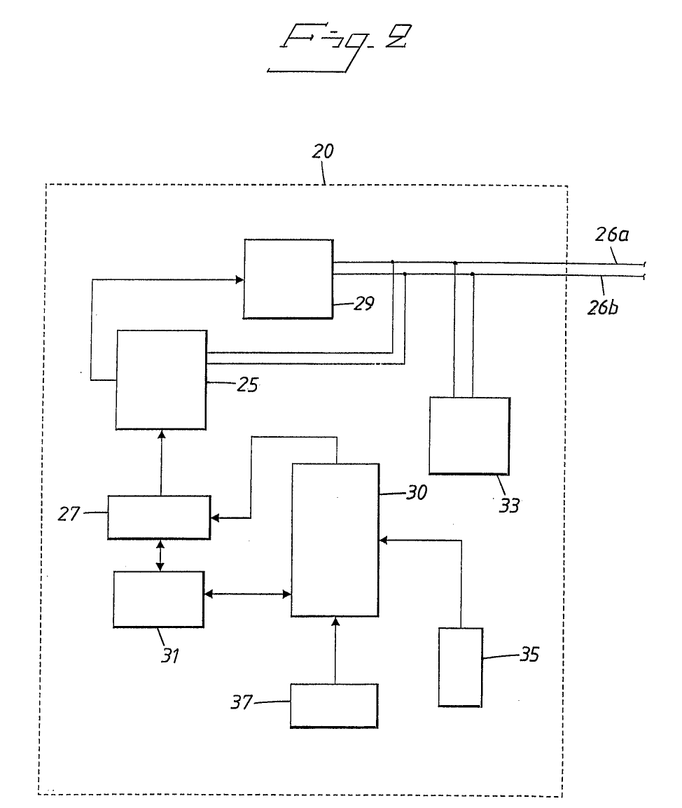

With reference now to FIG. 2, the configuration including the primary components of an embodiment of the present invention will be described. The illustrated embodiment includes an implantable medical device 20, such as the pacemaker shown in FIG. 1, and leads 26a and 26b, of the same type as the leads 6a and 6b shown in FIG. 1, for delivering signals between the implantable medical device 20. The leads 26a, 26b may be unipolar or bipolar, and may include any of the passive or active fixation means known in the art for fixation of the lead to the cardiac tissue. As an example, the lead distal tip (not shown) may include a tined tip or a fixation helix. The leads 26a, 26b have one or more electrodes (as described with reference to FIG. 1), such as a tip electrode or a ring electrode, arranged to, inter alia, transmit pacing pulses for causing depolarization of cardiac tissue adjacent to the electrode(-s) generated by a pacing pulse generator 25 under influence of a control circuit 27. The control circuit 27 controls pacing pulse parameters such as output voltage and pulse duration. Moreover, an impedance measuring circuit 29 is arranged to carry out the impedance measurements. The measuring impedance circuit 33 is arranged to apply excitation current pulses between any of the implanted electrodes 26a, 26b. The electrodes used for impedance measurement may be, for example, unipolar or bipolar electrodes located in or on the right atrium, the left atrium, the right ventricle or the left ventricle. Further, the pacemaker encapsulation is frequently used as an electrode for impedance measurements. The voltage measurements made by the impedance circuit may be between the electrodes used for current injection or between other electrodes. The electrodes used for impedance measurement are selected depending on the purpose of the impedance measurement. For intrathoracic measurements such as pulmonary edema monitoring it is essential to include tissue outside of the heart in the impedance measurement and in this case at least one electrode outside of the heart such as the pacemaker encapsulation should be used in the impedance measurement configuration. For monitoring of the heart such as cardiak stroke volume, diastolic and systolic slope etc.at least one electrode should be located inside the heart in the impedance measurement circuit. Further, the impedance measuring circuit 29 is coupled to a microprocessor 30, where processing of the obtained impedance signals can be performed. In an embodiment where the cardiac component of the electrical bio-impedance is sensed, the impedance measuring circuit 29 is arranged to apply an excitation current pulse between a first electrode and a second electrode arranged to be positioned at different position within the heart of the patient and to sense the impedance in the tissues between the first and second electrode to the excitation current pulse. The microprocessor 30 may be arranged to extract the cardiac component of the sensed impedance. This cardiac component can be used for calculating parameters like systolic and diastolic slopes, the pre-ejection period, or left ventricular ejection time. This calculation can be performed in accordance with conventional practice within the art.

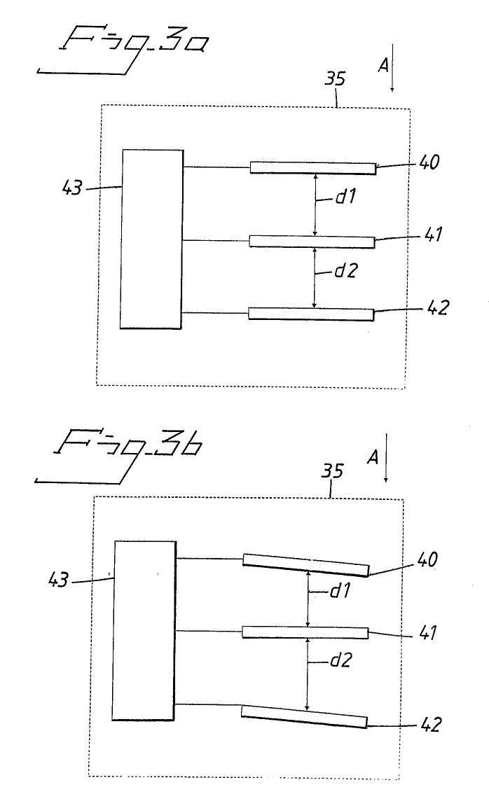

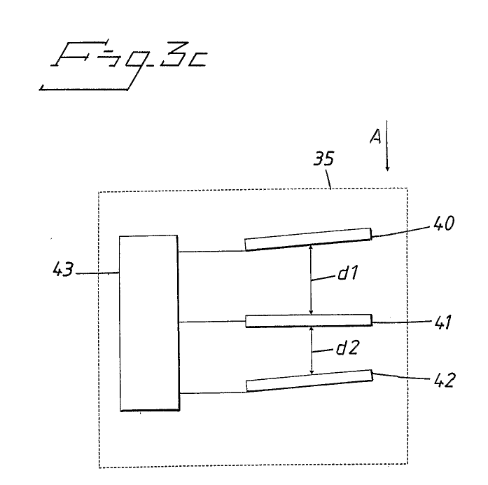

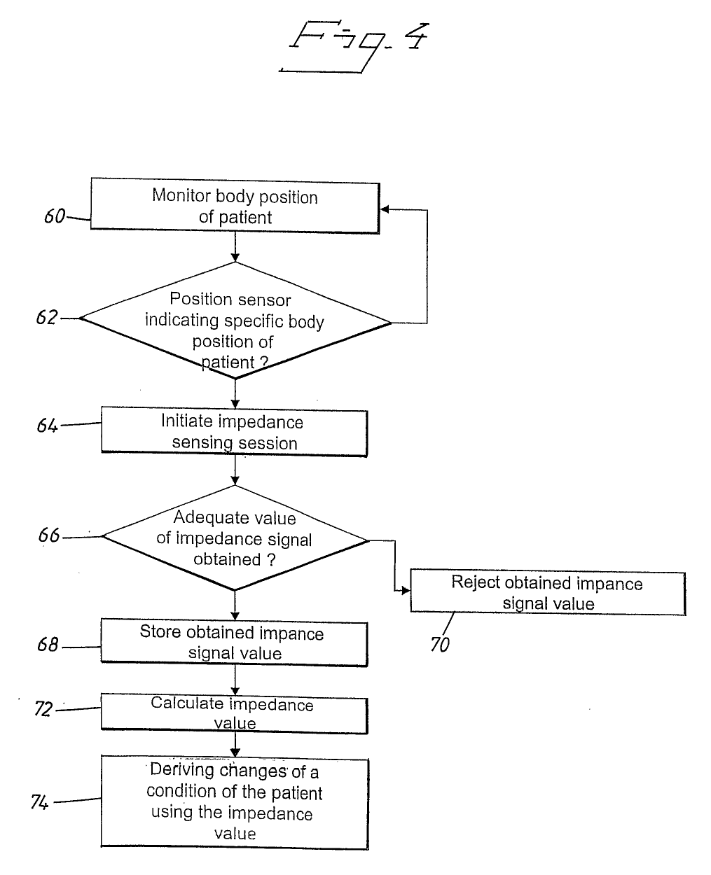

The impedance measuring circuit 29 is controlled by the microprocessor 30 and the control circuit 27. The control circuit 27 acts under influence of the microprocessor 30. A storage unit 31 is connected to the control circuit 27 and the microprocessor 30, which storage unit 31 may include a random access memory (RAM) and/or a non-volatile memory such as a read-only memory (ROM). Detected signals from the patients heart are processed in an input circuit 33 and are forwarded to the microprocessor 30 for use in logic timing determination in known manner. Furthermore, the implantable medical device 20 according to the present invention comprises position detecting sensor 35 arranged to detect a predetermined, specific body position of said patient. In a preferred embodiment of the present invention, the position detecting means is a back-position sensor arranged to sense when the patient is lying on his/hers back (or on his or hers face), see, for example, FIG. 3a. The position detecting sensor 35 is connected to the microprocessor 30. The implantable medical device 20 is powered by a battery 37, which supplies electrical power to all electrical active components of the medical device 20. Data contained in the storage unit 31 can be transferred to a programmer (not shown) via a programmer interface (not shown) for use in analyzing system conditions, patient information, calculation of surrogate parameters such as systolic and diastolic slopes, the pre-ejection period, or left ventricular ejection time and changing pacing conditions.

With reference now to FIG. 3a, 3b and 3c, a preferred embodiment of the position detecting sensor will be described. According to this embodiment, the position detecting sensor 35 includes a first conducting plate 40, a second conducting plate 41, and a third conducting plate 42, wherein the first and second plates 40 and 41 are spaced apart with a first distance d₁ and the second and third plates 41 and 42 are spaced apart with a second distance d₂, see FIG. 3a. Each plate 40, 41, 42 is connected to a discriminating circuit 43 arranged to sense a first capacitance c₁ between the first and second plates 40 and 41, respectively, and a second capacitance c₂ between the second and third plates 41 and 42, respectively. According to one embodiment, the first and second capacitor plates 40 and 42 are flexible. In another embodiment, the first and second capacitor plates 40 and 42 are pivotally suspended. Preferably, the first and second capacitor plates 40 and 42 are arranged to, when the sensor is positioned such that the plates 40-42 are substantially parallel with ground, will move, i.e. bend or pivot, slightly against the ground under the influence of gravity. Thereby, the first and second distance d₁ and d₂ will change and there will, in turn, arise a difference between the first capacitance c₁ and c₂, which can be sensed by the discriminating circuit 43. When the first distance d₁ is shorter than the second distance d₂, the first capacitance c₁ will be larger than the second capacitance c₂, see FIG. 3b. In this case the sensor is arranged to deliver a positive signal, c₁-c₂. Inversely, when the first distance d₁ is longer than the second distance d₂, the first capacitance c₁ will be smaller than the second capacitance c₂, see FIG. 3c. Accordingly, the sensor will deliver a negative signal c₁-c₂.

Moreover, in this embodiment, the first and second distance d₁ and d₂ are equal and the plates 40-42 are arranged so that the first capacitance c₁ is equal to the second capacitance 2 when the sensor is positioned such that the capacitor plates 40-42 are perpendicular or forming an angle with respect to the ground. Consequently, when the patient is in positions such that the capacitor plates 40-42 are perpendicular or forming an angle with respect to the ground, the sensor 35 will not deliver any signal since c₁, is equal to c₂.

Preferably, the sensor is installed in an implantable medical device such that there will arise a difference between c₁ and c₂ when the patient carrying the device lies on his or her back (or on his or hers face), due to the fact that plates 40 and 42 are positioned substantially parallel to the ground and therefore will move, i.e. bend or pivot, against ground, and such that the plates 40 and 42 are not affected by the gravity when the patient is in other positions, for example, lying on his or hers side or standing. For example, when the patient is lying on his or hers back, the sensor is arranged such that the first plate 40 and the second plate 42 will bend in the direction indicated by the arrow A, thereby the first distance d₁ will be shorter than the second distance d₂ and the first capacitance c₁ will be larger than the second capacitance c₂, see FIG. 3b. In this case, the sensor is arranged to deliver a positive signal, c₁-c₂. Inversely, when the patient is lying on the face, the first plate 40 and the second plate 42 will bend in a direction against the arrow A, the first distance d₁ will be longer than the second distance d₂ and the first capacitance c₁ will be smaller than the second capacitance c₂, see FIG. 3c. Accordingly, the sensor will deliver a negative signal c₁-c₂. Thus, the position sensor 35 is capable of discriminating between different horizontal positions of the patient.

Referring now to FIG. 4, a detailed description of the method according to the present invention will be given. At step 60, the position sensor 35 monitors or detects the position of the patient in order to detect a predetermined specific body position of the patient, i.e. the sensor is arranged to supply a position indicating signal when the patient is in the specific position as described above. In a preferred embodiment, the specific predetermined body position is when the patient is lying on the back (or on the face). During periods when the patient is in other positions than the predetermined specific position, the impedance measuring circuit 29 is in an idle mode. When the patient is the specific body position, the sensor, in step 62, supplies a position indicating signal or triggering signal to the microprocessor 30. The microprocessor influences the control circuit 27, which, in turn, puts the impedance measuring circuit 29 in an active mode where the measuring circuit 29 initiates an impedance measuring session, which will be described below. Thereafter, at step 66, it is judged whether the obtained impedance signals value is valid. This can be performed, for example, by checking that the obtained value is within a preset range including the preceding value. If the obtained impedance signal is found to be valid, it is stored temporarily, at step 68, in the storage means 31. If the obtained value is found to be invalid, i.e. the value being outside the preset range, the signal is rejected. In one embodiment, an new impedance measuring session is initiated after a delay period of a predetermined length and if this is repeated a preset number of times without obtaining a valid signal the impedance measuring circuit returns to the idle mode. At step 72, the stored impedance signals is used to calculate impedance values. This calculation can be performed through execution of suitable software in the microprocessor 30. Thereafter, at step 74, the calculated values is compared with stored impedance values obtained in earlier impedance measuring sessions in order to monitor, for example, changes and/or trends of the development of the impedance. In this manner, it can be derived whether a condition of the patient influencing the impedance is changing, for example, congestive heart failure.

As mentioned above, electrical bio-impedance signals has been found to constitute an effective measure for identifying changes of many different conditions in the body of a patient. According to a preferred embodiment, the obtained impedance signals are utilized to monitor or detect incipient pulmonary edema and the progression of pulmonary edema due to CHF. Since the accumulation of fluids in the lung-region associated with pulmonary edema affects the thoracic impedance, or more specifically the DC impedance level, due to the fact that the resistivity of the lung changes in accordance with a change of the ratio of fluid to air, trends and/or changes of the impedance levels constitute a useful measure in order to monitor or detect incipient edema. The DC impedance level is negatively correlated with the amount of fluids in the lung. There are a number of possible impedance configurations, i.e. ways of injecting current between two electrodes in the pacemaker and then to measure the voltage the current provokes between the electrodes. For example, impedance configurations can be unipolar, bipolar, tripolar or quadro-polar. The configuration known as bipolar means, in practice, a configuration where the current and the voltage is sent out and measured between the same two electrodes. When one of the electrodes used in a bipolar measurement is the housing or the case, the configuration is called unipolar. For example, in FIG. 1, between the housing of the pacemaker 2 and a right ventricular electrode arranged at the distal end of lead 6a. A tri-polar configuration uses three electrodes, i.e. the current injection and the voltage measurement share one electrode. As an example, the current can be sent out from the housing or the case of the medical device to a RV-tip and the voltage is measured between the case and RV-ring. In quadro-polar measurements, the current is sent out between electrodes and the voltage is measured between two entirely different electrodes, i.e. in this case there are four electrodes involved.

According to embodiments of the present invention, different measurements conditions can be specified in order to obtain more accurate impedance values. For example, the initiation of the impedance measuring session can be delayed a predetermined period of time, for example 0-10 h, after that the signal indicative that the patient is in the specific position. Furthermore, according to another embodiment of the present invention, a condition for initiating the impedance measuring session is that a sensed activity level of the patient is within a predetermined range. The activity level can be sensed be means of an activity sensor incorporated in the medical device in accordance with conventional practice within the art. That is, even if the patient is in the specific position, the impedance measuring session is initiated only if the activity level signal is within the predetermined range.

As mentioned above, also electrical bio-impedance signals has been found to constitute an effective measure for identifying changes of many different conditions in the body of a patient, and according to one embodiment of the present invention, the cardiac component of the impedance measured between electrodes within the heart is used to calculate surrogates for heart failure. Thus, by monitoring or detecting trends and/or changes of these surrogates, for example, parameters, such as the systolic and diastolic slopes, pre-ejection period and left ventricular ejection time, progress of conditions such as CHF can be studied. The cardiogenic impedance is defined as the impedance or resistance variation that origins from cardiac contractions measured by electrodes inside or on the surface of the body. The impedance is calculated as z=u/i, where u is the measured voltage between two electrodes and i is the applied excitation current between the two electrodes. Normally, the electrodes are placed inside or on the surface of the heart, integrated on a pacemaker lead, for example the leads 6a, 6b shown in FIG. 1. The cardiogenic impedance variation correlates to the volume changes of the heart chambers, which can be used as an indication of the dynamic blood filling. Preferably, the microprocessor 30 is arranged to filter the cardiac component from the obtained electrical bio-impedance and to extract systolic and diastolic slopes, the pre-ejection period, or left ventricular ejection time using the data corresponding to the cardiac component of the bio-impedance signals obtained in the impedance measuring session. In addition, this extracting procedure can be performed in an external unit, wherein the filtered cardiac component is transferred from the medical device via the telemetry device (not shown).

According to embodiments of the present invention, different measurements conditions can be specified in order to obtain more accurate impedance values. As an example, the impedance measurements can be correlated with the heart rate of the patient. For this purpose, the heart rate of the patient is sensed and it is determined whether the sensed heart rate is within a predetermined range, and the impedance measuring session is initiated only if the heart rate is within the predetermined range. That is, even if the patient is in the specific position the impedance measuring session is initiated only if the heart rate is within the predetermined range. In this embodiment, means for sensing the heart rate of the patient is incorporated in the medical deice in accordance with conventional practice within the art.

Although an exemplary embodiment of the present invention has been shown and described, it will be apparent to those of ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims.

We claim as our invention:

1.-37. (canceled)

38. A method for detecting a change in a medical condition of a patient, comprising the steps of:
determining that a patient satisfies specified criteria ensuring comparability of measurements results obtained from the patient;
only when said patient satisfies said specified criteria, obtaining electrical bio-impedance signals from the patient respectively as separated points in time;
extracting a cardiac component of each electrical bio-impedance signal, said cardiac component of each electrical bio-impedance signal being associated with a medical condition of the patient; and
analyzing said cardiac components of the respective electrical bio-impedance signal to identify a change in said medical condition.

39. A method as claimed in claim 38 comprising determining that the patient satisfies said specified criteria by determining when the patient is in a specified body position.

40. A method as claimed in claim 39 comprising, after determining that the patient is in said specified body position, delaying obtaining said electrical bio-impedance signal from the patient for a predetermined period of time.

41. A method as claimed in claim 38 comprising determining that the patient satisfies said specified criteria by sensing a heart rate of the patient, determining whether the heart rate is a within a predetermined range, and obtaining said electrical bio-impedance signal from the patient only if the heart rate is within said predetermined range.

42. A method as claimed in claim 38 comprising determining that the patient satisfies said specified criteria by sensing a activity level of the patient, determining whether the activity level is a within a predetermined range, and obtaining said electrical bio-impedance signal from the patient only if the activity level is within said predetermined range.

43. A method as claimed in claim 38 comprising electronically storing the respective electrical bio-impedance signals.

44. A method as claimed in claim 38 wherein the step of obtaining said electrical bio-impedance signals from the patient comprises measuring an intrathoracic impedance of the patient.

45. A method as claimed in claim 44 wherein the step of measuring the intrathoracic impedance of the patient comprises:
applying an excitation current pulse between a first electrode located within the heart of the patient, and a second electrode; and
measuring said electrical bio-impedance in tissue located between said first electrode and said second electrode in response to said excitation current pulse.

46. A method as claimed in claim 44 comprising analyzing said cardiac components of the respective electrical bio-impedance signals to detect insipient pulmonary edema.

47. A method as claimed in claim 38 comprising extracting said cardiac component of each electrical bio-impedance signal by:
applying an excitation current pulse between a first electrode located at a first position within the heart of the patient and a second electrode located at a second, different position within the heart of the patient;
measuring impedance in tissue between said first and second electrodes in response to said excitation current pulse; and
extracting the cardiac component of said impedance in said tissue.

48. A method as claimed in claim 38 comprising, from said cardiac component of the electrical bio-impedance signal, extracting a systolic slope and a diastolic slope.

49. A method as claimed in claim 38 comprising, from said cardiac component of said electrical bio-impedance signal, extracting an identification of the pre-ejection period.

50. A method as claimed in claim 38 comprising, from said cardiac component of said electrical bio-impedance signal, extracting an identification of the left ventricular ejection time.

51. A medical device for detecting a change in a medical condition of a patient, comprising:
a position detector configured to interact with a patient to identify when the patient is in a predetermined body position, said position detector emitting a trigger signal when the patient is in said predetermined body position;
an impedance measuring arrangement connected to said position detector and supplied with said trigger signal, said impedance measuring arrangement, in response to each of a plurality of trigger signals emitted by said position detector at respective, separated points in time, obtaining an electrical bio-impedance signal from the patient;
an analysis unit that extracts the cardiac component from each of the electrical bio-impedance signals obtained by the impedance measuring arrangement, said cardiac component being associated with a medical condition of the patient; and
an analysis unit that analyzes the respective cardiac components to identify a change in said medical condition from the respective cardiac components.

52. A medical device as claimed in claim 51 wherein said position detector emits said trigger signal when said position detector detects that the patient is lying on the patient's back.

53. A medical device as claimed in claim 51 comprising a memory connected to said impedance measuring arrangement that stores said electrical bio-impedance signals.

54. A medical device as claimed in claim 51 wherein said impedance measuring arrangement is configured to measure transthoracic impedance of the patient.

55. A medical device as claimed in claim 54 wherein said impedance measuring arrangement comprises:
a first electrode configured to be positioned within the heart of the patient;
a second electrode;
a current source connected to said first electrode and said second electrode that applies an excitation current pulse between said first electrode and said second electrode; and
a measuring unit that measures impedance in tissue between said first electrode and said second electrode in response to said excitation current pulse.

56. A medical device as claimed in claim 54 wherein said analysis unit detects insipient pulmonary edema as said medical condition.

57. A medical device as claimed in claim 51 wherein said impedance measuring arrangement comprises:
a first electrode configured to be positioned at a first position within the heart of the patient;
a second electrode configured to be positioned at a different, second position within the heart of the patient;
a current source connected to said first electrode and to said electrode that applies an excitation current pulse between said first electrode and said second electrode; and
a measuring unit that measures impedance in tissue between said first electrode and said second electrode in response to said excitation current pulse.

58. A medical device as claimed in claim 57 comprising:
a heart rate detector configured to interact with the patient to detect the heart rate of the patient and to generate a signal indicative of said heart rate; and
a processor supplied with said signal generated by said heart rate detector, said processor, upon receiving said signal, determining whether said heart rate is within a predetermined range and, if so, enabling said impedance measuring arrangement to obtain said electrical bio-impedance signal.

59. A medical device as claimed in claim 58 wherein said position detector supplies said trigger signal also to said processor, and wherein said processor, upon receiving said trigger signal, causes said impedance measuring arrangement to delay obtaining said electrical bio-impedance signal from the patient for a predetermined period of time.

60. A medical device as claimed in claim 58 comprising an activity sensor configured to interact with the patient to detect an activity level of the patient and to generate a signal indicative of said activity level, and wherein said processor is supplied with said signal generated by said activity sensor and determines whether said activity level is within a predetermined range and, if so, enables said impedance measuring arrangement to obtain said electrical bio-impedance signal.

61. A medical device as claimed in claim 51 wherein said analysis unit further extracts a systolic slope and a diastolic slope from said cardiac component.

62. A medical device as claimed in claim 51 wherein said analysis unit determines the pre-ejection period from said cardiac component.

63. A medical device as claimed in claim 51 wherein said analysis unit determines the left ventricular ejection time from said cardiac component.

64. A computer-readable medium encoded with a data structure, said computer-readable medium being loadable into a controller of a medical device for causing said medical device to:
determine that a patient satisfies specified criteria ensuring comparability of measurements results obtained from the patient;
only when said patient satisfies said specified criteria, obtain an electrical bio-impedance signal from the patient;
extract a cardiac component of the electrical bio-impedance signal, said cardiac component of the electrical bio-impedance signal being associated with a medical condition of the patient; and
analyze said cardiac component of said electrical bio-impedance signal to identify a change in said medical condition.
